(19) United States
(12) Patent Application Publication
(10) Pub. No.: US 2016/0367214 A1
(43) Pub. Date: Dec. 22, 2016

(54) FETAL HEART RATE MONITORING ASSEMBLY

(71) Applicant: Laerdal Medical AS, Stavanger (NO)

(72) Inventors: Helge MYKLEBUST, Stavanger (NO); Helge FOSSAN, Stavanger (NO); Joar EILEVSTJØNN, Sandnes (NO); Cenk AYTKEN, Stavanger (NO)

(21) Appl. No.: 14/742,138

(22) Filed: Jun. 17, 2015

(51) Int. Cl. A61B 8/02; A61B 5/00; A61B 5/024

(52) U.S. Cl. CPC A61B 8/02; A61B 5/02438; A61B 5/4362; A61B 5/742; A61B 2503/02

(57) ABSTRACT

A fetal heart rate measuring assembly having a transducer housing with an ultrasound transducer. The transducer housing is adapted to be coupled to the abdomen of a pregnant woman. The assembly has display unit comprising a display, and a wire extending between the transducer housing and the display unit. The assembly further comprises a dry electrode pair, by means of which the fetal heart rate measuring assembly is adapted to measure the heart rate of the pregnant woman. The dry electrode pair is arranged on the display unit.

The present invention relates to a monitoring assembly adapted to monitor heart rates of a fetus. In particular the monitoring assembly is adapted for such monitoring during labor in low resource environments, typically maternity units having few midwifes and small resources.

### BACKGROUND

Each year approximately 136 million babies are born globally, and it is estimated that about 90% make the transition from intrauterine to extra uterine life without any intervention. The remaining 13.6 million new-borns are delivered with absent or poor respiratory effort and need some degree of support to achieve cardiopulmonary stability. Between 3-6% need assisted positive pressure ventilation, and less than 1% require advanced resuscitation including intubation, chest compressions, and medications.

Intrapartum hypoxia (often equated with birth asphyxia) is estimated to account for about two million perinatal deaths annually including intrapartum stillbirths and early neonatal deaths, with 98-99% of the burden in low and middle income countries. An additional one million of the surviving infants develop neurocognitive problems such as cerebral palsy and learning difficulties.

The incidence of intrapartum-related stillbirths and neonatal deaths has remained essentially unchanged over the past 15 years. The lack of impact on neonatal mortality rates the first day of life is a major concern.

US20120232398 discloses a wireless fetal monitoring system. It suggested to stick a patch on the mothers belly, the patch including EMG electrodes and an ultrasound sensor array. The patch is adapted for both fetus and mother monitoring.

Moreover, WO9615713 suggests discriminating fetus measurements from mother measurements. Additional sensors are placed on the body of the mother, however away from the fetus. To position such sensors in contact with the mother body, they are sewn into an undergarment that closely fits over a portion of the mother body. This publication also teaches about the risks of using ultrasound technique to measure the fetus.

WO2015005796 discloses a fetal heart rate monitoring assembly, comprising an ultrasound transducer adapted for measurement of fetal heart rate. It further comprises a display unit, which is carried by the pregnant woman, in a harness around her neck.

Solutions of the prior art tend to be suited for the high-end market, where supply of single use equipment and their related cost is not a concern. Such products may typically be patches that are fastened to the mother body with adhesive. Moreover, the solutions are adapted for an environment having a high number of medical personnel. The object of the present invention is to provide a fetal heart rate measuring assembly which is more suited for a low resource market, where supply of single use equipment is unreliable. Moreover, the present inventions seeks to provide a solution where also the pregnant mother may be used as a resource.

### THE INVENTION

According to the present invention there is provided a fetal heart rate measuring assembly comprising a transducer housing which is equipped with an ultrasound transducer. The ultrasound transducer is adapted to be coupled to the abdomen of a pregnant woman. The assembly further comprises a display unit with a display. A wire connects the transducer housing and the display unit. The fetal heart rate measuring assembly further comprises a dry electrode pair, with which the fetal heart rate measuring assembly is adapted to measure the heart rate of the pregnant woman. The dry electrode pair is arranged on the display unit.

With the term “dry electrode”, is meant that it may be used without applying any type of fluid or gel in order to obtain a measurement of acceptable quality. This is in contrast to many prior art solutions, where electrodes are used to produce cardiograms that not only contains heart rate, but also additional information.

With such a fetal heart rate measuring assembly, an assembly is provided with does not require single use accessories. Moreover, it is suited for making the pregnant woman participate in obtaining correct fetal heart rate measurements.

Advantageously, two separate dry electrodes of the dry electrode pair are arranged with a distance between them. Preferably, when in use, the pregnant woman may engage her respective hands to one respective dry electrode of the dry electrode pair.

Advantageously, the fetal heart rate measuring assembly comprises logic circuitry which is configured to compare a measured mother heart rate, as measured with the dry electrode pair, with a heart rate measured with the ultrasound transducer.

Preferably, the logic circuitry is configured to determine a similarity value for the two measured heart rates, and to provide an alert when the determined similarity value is above a preset value. Such an alert may typically be in the form of a symbol appearing on the display. The alert notifies, for instance to a midwife, that the two measured heart rates have such a similarity that there is a possibility that they are both representing the heart rate of the mother. The midwife may then, typically, alter the position of the transducer housing in order to obtain a correct measurement of the fetal heart rate. If successful, in a normal circumstance the logic circuitry will determine a similarity value which is below the preset value, and the alert will be removed/stopped.

In a preferred embodiment, the dry electrode pair is arranged on a front panel of the housing of the display unit. For instance, one dry electrode may be arranged on respective, opposite side of the display.

### Example of Embodiment

Having described the present invention in general terms above, a more detailed example of embodiment will be described in the following with reference to the drawings, in which

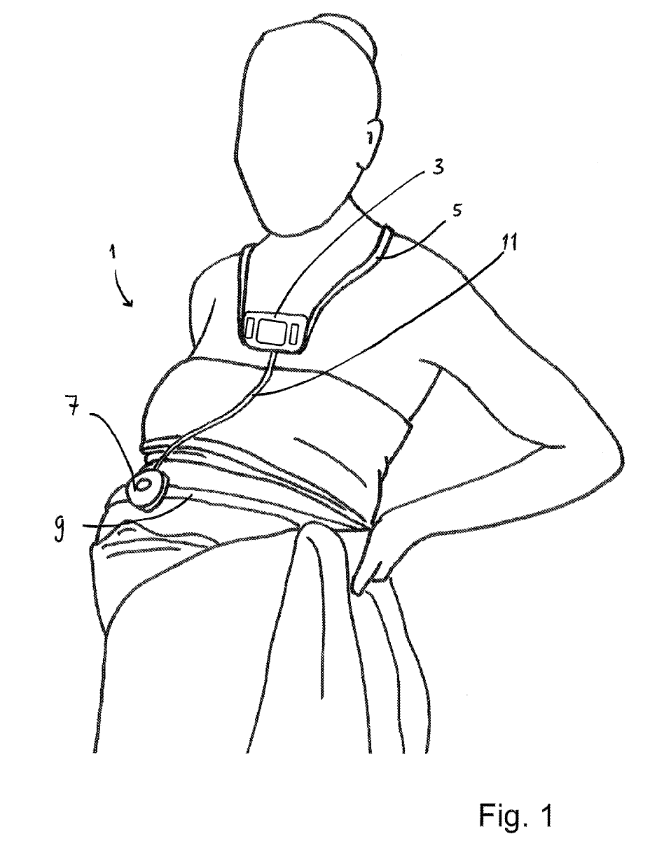

FIG. 1 illustrates a FHR measuring assembly according to the invention being carried by a pregnant woman;

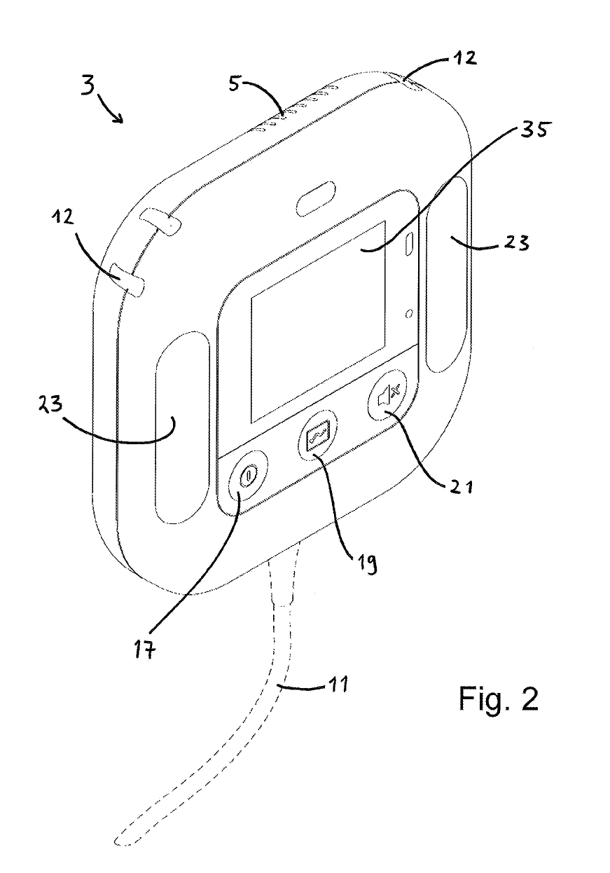

FIG. 2 is a perspective view of a display unit, which is a part of the assembly shown in FIG. 1;

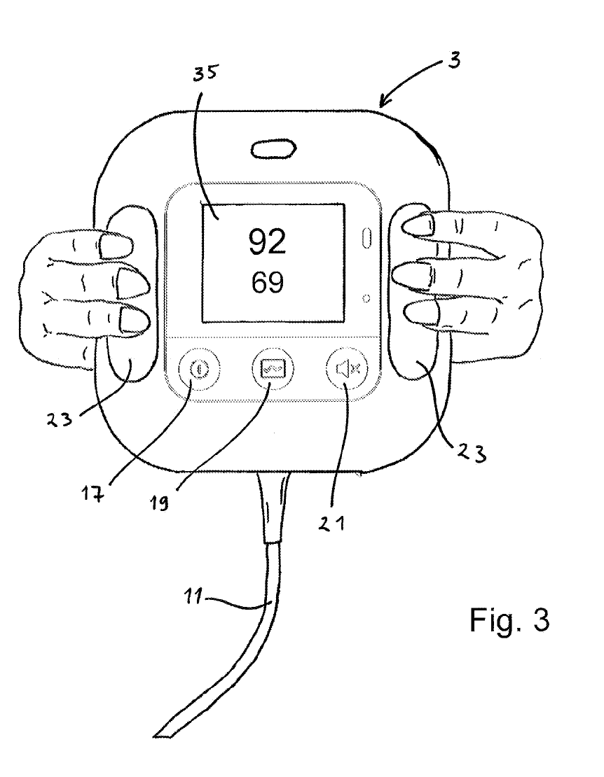

FIG. 3 is a front view of the display unit which is being held by the pregnant woman at the position of two dry electrodes; and

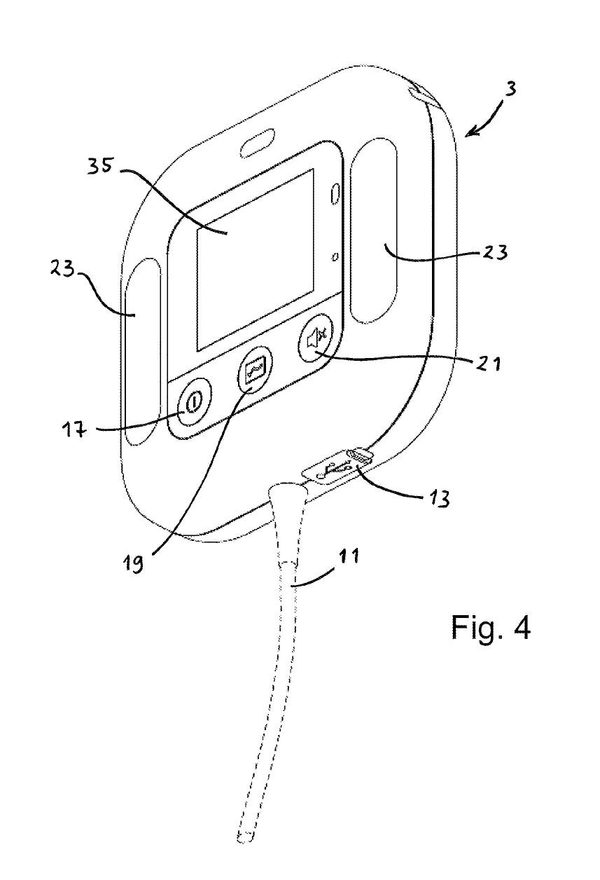

FIG. 4 is another perspective view of the display unit, seen from below.

FIG. 1 illustrates a pregnant woman having a fetal heart rate measuring assembly 1 arranged to her. A display unit 3 is loosely hung around the neck of the woman by means of a harness 5. A transducer housing 7 is strapped onto the abdomen of the woman by means of a strap 9. A wire 11 connects the display unit 3 to the transducer housing 7. The transducer housing 7 has an ultrasound transducer adapted to measure the fetal heartbeat.

FIG. 2 depicts the display unit 3 shown in FIG. 1, however with a somewhat different design. The display unit 3 is adapted to present the measured fetal heartbeat on a display 35 showing the current or last measured heartbeat of a fetus. It also comprises an audio communication means 15. The audio communication means 15 is typically a small loudspeaker which can give a warning signal or other types of audio signals.

On two oppositely arranged upper edges of the housing of the display unit 3, there is arranged a harness connection section 12.

On the front panel of the display unit 3, three operation buttons are arranged. These include an on-and-off switch 17, a historic data button 19, and a mute button 21. The on-and-off switch 17 is used to turn the fetal heart rate measuring assembly 1 on and off. The historic data button 19 is used to make the display unit 3 present recorded data. Typically, the display 35 may then present a curve representing earlier measurements of the fetal heart rate. The mute button 21 can be pressed, typically by a midwife or other medical personnel, in order to silence an alarm. Such an alarm may typically be given by the audio communication means 15 if the measured fetal heart rate has fallen below an alarm setpoint.

A problem which may arise when using the transducer housing 7 to measure the fetal heart rate, is that the assembly may not be able to distinguish between the heart rate of the mother and the fetal heart rate. This problem may be due to a suboptimal positioning of the transducer housing 7 and/or poor acoustic coupling between the transducer housing 7 and the abdomen of the woman. In the prior art, it is known to measure both the fetus heart rate and the mother heart rate, and to distinguish between the two with a logic circuit. However, constant monitoring of the mother heart rate according to the solutions of the prior art increases the number of sensors which need to be coupled to the mother body.

Still referring to FIG. 2, the display unit 3 comprises a dry electrode pair which has two dry electrodes 23. The two dry electrodes 23 are permanently installed in the display unit 3. In some cases, it may happen that the fetal heart rate measuring assembly 1 will, unintentionally, measure a combination of the fetal heart rate and the mother heart rate with the ultrasound transducer. Or, it may happen that the only the mother heart rate is measured with the ultrasound transducer. In such cases, an alarm (preferably a soft alarm) may be produced, signaling that the measurement is not correctly performed. For instance, the alarm may be a symbol on the display 35. To ensure correct measurements, the pregnant woman may then press her finger tips onto the respective dry electrodes 23, so that the fetal heart rate measuring assembly 1 can measure the mother heart rate, without risk of confusing the heart rate with the fetal heart rate. The assembly may compare the mother heart rate, as measured with the two dry electrodes 23, with the heart rate measured with the ultrasound transducer (which may be fetal and/or mother heart rate). Such comparison can be performed with logic circuitry which may produce a similarity value that represents the similarity of the two measured heart rates. If the similarity value remains above a preset value over some time (for instance 10-30 seconds), the heart rate measured with the ultrasound transducer is most likely the heart rate of the pregnant woman. In such a situation, the alarm is provided and the midwife will be triggered to reposition the ultrasound transducer. If the new position of the ultrasound transducer is functioning (measuring the fetal heart rate), the similarity value will be determined to be below the preset value.

Embodiments without the alarm may also be used, since the two measured heart rates may be displayed simultaneously on the display 35. It will then be easy for the midwife to perform a comparison and to evaluate the similarity.

FIG. 3 illustrates the display unit 3 in a situation where the mother presses three finger tips against each of the two dry electrodes 23. As a result, the fetal heart rate measuring assembly 1 presents both the mother heart rate (69/min) and the fetus heart rate (92/min) on the display 35.

FIG. 4 shows the display unit 3 in a perspective view, seen from below. In the bottom face of the display unit 3, next to the connecting interface between the wire 33 and the display unit housing, an USB connection interface 13 is shown. The USB connection interface 13 can be used to charge a battery within the display unit 3 and to output measurement data from a storage unit within the display unit 3. One may also use the USB connection interface 13 to setup/program various parameters/functions of the fetal heart rate measuring assembly 1. For instance, one may set the fetal heart rate measuring assembly 1 into a training mode, for use during midwife training.

Although the fetal heart rate assembly 1 advantageously will be programmed for performing heart rate measurements at given intervals, e.g. every 5, 10, or 15 minutes, it is preferably also able to perform a measurement on demand. For instance, after having repositioned the pregnant woman, one may want to perform a measurement at once without waiting for the next scheduled measurement. Such a non-scheduled measurement may be initiated by manipulating shown buttons 17, 19, 21, or another additional button may be present on the display unit 3. Continuous fetal heart rate may also be chosen, meaning that the fetal heart rate measurement number is updated for instance about every 1 to 2 second.

In one embodiment, the fetal heart rate measuring assembly 1 may be provided with software or electric circuitry which is configured to indicate how long the pregnant woman should touch the two dry electrodes 23. Accordingly, it may be indicated on the display that the pregnant woman should keep her hands on the dry electrodes 23, or if she may let go. For instance, in a situation where the distinction of the two heart rates is quickly determined by the assembly, an indication can be provided, that she may let go of the electrodes. Contrary to this, if the heart rates are similar, or if there is a poor acoustic connection between the transducer and the abdomen of the pregnant woman, the assembly may need more time to perform a comparison of the two heart rates.

The assembly 1 may also be configured to provide an indication on the display, instructing the midwife to reposition the transducer.

1. A fetal heart rate measuring assembly comprising a transducer housing with an ultrasound transducer, adapted to be coupled to the abdomen of a pregnant woman, a display unit comprising a display, and a wire extending between the transducer housing and the display unit, wherein the fetal heart rate measuring assembly further comprises a dry electrode pair, with which the fetal heart rate measuring assembly is adapted to measure the heart rate of the pregnant woman, wherein the dry electrode pair is arranged on the display unit.

2. A fetal heart rate measuring assembly according to claim 1, wherein two dry electrodes of the dry electrode pair are arranged with a distance between them.

3. A fetal heart rate measuring assembly according to claim 1, wherein it comprises logic circuitry which is configured to compare a measured mother heart rate, as measured with the dry electrode pair, with a heart rate measured with the ultrasound transducer.

4. A fetal heart rate measuring assembly according to claim 3, wherein said logic circuitry is configured to determine a similarity value for the two measured heart rates, and to provide an alert when the determined similarity value is above a preset value.

5. A fetal heart rate measuring assembly according to claim 2, wherein the dry electrode pair is arranged on a front panel of the housing of the display unit, one dry electrode being arranged on respective opposite sides of the display.
